(19) United States
(12) Patent Application Publication
(10) Pub. No.: US 2012/0189490 A1
(43) Pub. Date: Jul. 26, 2012

(54) CLEANSING SYSTEM USING OZONE AND NEBULIZED FLUIDS

(76) Inventors: Jozef Van den bossche, Londerzeel (BE); Frank Bakker, Vught (NL)

(21) Appl. No.: 13/328,791

(22) Filed: Dec. 16, 2011

Related U.S. Application Data
(60) Provisional application No. 61/435,596, filed on Jan. 24, 2011.

(51) Int. Cl. A61L 9/015; B01J 19/12

(52) U.S. Cl. 422/5; 422/186.07; 422/119

(57) ABSTRACT

A process is described for cleansing and deodorizing an environmental space including deodorizing a space with an ozone treatment for a pre-selected time; and thereafter, automatically and without human intervention, exposing the space to a nebulized liquid, having ozone quenching properties and comprising an odor neutralizing or air freshening agent, to reduce ozone levels to a level safe for human exposure and to remove residual odors. An apparatus for carrying out the process and compositions for use in the process are further disclosed.

### RELATED APPLICATIONS

This application is a non-provisional application that claims priority benefit of U.S. Provisional Application Ser. No. 61/435,596 filed 24 Jan. 2011, the contents of which are presented herein by references.

### FIELD OF THE INVENTION

The present invention relates to the cleansing, disinfection and deodorization of both indoor and outdoor environments. More specifically, the present invention provides a system to degrade odorous materials through the use of ozone and to deliver further cleansing via the action of odor neutralizers, disinfectants, or both, which may also act to quench the levels of ozone present in the environmental space to be treated.

### BACKGROUND

It is estimated that people spend approximately ninety percent of their time indoors. Indoor pollution sources that release gases or particles into the air are the primary causes of poor indoor air quality. Inadequate ventilation increases indoor pollutant levels by not passing enough outdoor air into the space to dilute emissions from indoor sources, as well as failing to purge indoor air pollutants from any confined spaces.

Odor problems originate from numerous sources: bacteria, molds, tobacco, smoking, fumes from chemicals, cooking, fireplaces, and pets. The contamination from mold and fungus is another major source of unpleasant odors.

The U.S. Environmental Protection Agency cites three main strategies for reducing indoor air pollutants: source control, ventilation, and air cleaning. Source control is considered the most effective and eliminates the sources of pollutants or reduces their emissions. Regrettably, not all pollutant sources are readily identified and practically reduced. In an animal facility, for example, because of the large amount of noxious substances excreted, it is not economical to use source control such as absorption for deodorization of animal facilities. Ventilation is effective because it brings outside air indoors. Limitation on the use of ventilation centers around the costs for heating or cooling incoming air. Therefore, the most practical method for reducing indoor air pollutants rests on the use of air cleaning applications. One of such applications is through the use of ozone technology.

Ozone occurs naturally in the atmosphere and is a powerful oxidant. As it oxidizes a substance through ozonolysis, ozone breaks the substance molecule's covalent bonds. Ozone is able to oxidize organic substances such as bacteria and mildew, sterilize the air, and destroy odors and toxic fumes.

Ozone is produced from oxygen as a result of electrical discharge or ultraviolet (UV) radiation. Singlet oxygen atoms are formed by splitting of diatomic oxygen molecules to produce ozone molecules. Ozone produced for commercial application is generated by corona discharge, UV radiation, and electrolysis.

Ozone used for aerial treatment is typically conducted in gaseous form. Ozone consists of an oxygen molecule containing three atoms instead of two. The extra atom of ozone is known as a loose radical that looks for organics to attach to and thereby oxidize. Ozone is known as a friendly oxidizer, due to the fact that ozone reverts back to oxygen after oxidation occurs. Ozone is an oxidizing gas that travels throughout the room and oxidizes most organics and in the process, ozone neutralizes most odors and certain gases.

Masaoka et al. (Masaoka et al. Ozone decontamination of bioclean rooms, Applied and Environmental Microbiology, 43 (3), 509-513, 1982), discloses an application of decontaminating hospital bioclean rooms using ozone. This reference points out that ozone has proved to be a good decontaminant of test organisms at 40 ppm for 3 days. In comparison to the traditional administration of formaldehyde treatment, ozone application seems to be much easier to use, carries less after-use products (ozone gets converted to non-toxic oxygen once in the air), and decreases the inhalation of disinfectants by hospital staff.

Pan et al discloses the efficiency of ozonizing apparatus in removing ammonia in animal facilities (Pan et al., Deodorization of laboratory animal facilities by ozone, Exp. Anim. 44(3), 255-259, 1995). The apparatus cited in this reference takes in room air and allows it to react with a small amount of ozone gas which is generated by electric discharge and substances emitting bad smells are degraded by an oxidizing effect of ozone. However, this reference does not seem to mention a coupled mechanism whereby nebulized odor neutralizers deliver further treatment of both: a) odors not eliminated by ozone application, and b) odors newly introduced by ozone application.

The atomization of liquids induced by ultrasonic oscillation is equally known. Applications range from fountains used as decorative elements to humidifiers and nebulizers used for medicinal purposes such as inhalation devices in asthma treatment.

Barreras et al. (Barreras et al., Transient high-frequency ultrasonic water atomization, Experiments in Fluids, 33, 405-413, 2002) illustrates the relationship between physical properties of the fluid and the particle diameter of the aerosols generated by ultrasonic frequencies. The reference cites an apparatus which generates an aerosol stream to be able to penetrate all parts of a car air conditioning system without disassembling physical barriers such as pollen filters.

One of the advantages of ozone technology is its relatively low cost in comparison to other technologies. In addition, ozone is also less corrosive to materials and equipment than most chemicals currently being used such as chlorine. Further, ozone kills bacteria within a few seconds by a process known as cell lysing and therefore, micro-organisms tend not to develop ozone resistant strains. Finally, ozone technology is applied with very little, if any, manpower. Studies have been conducted to evaluate ozone fumigation as one of the new methods for the sanitation of odorous facilities such as animal rooms (Pan et al., Deodorization of laboratory animal facilities by ozone, Exp. Anim. 44(3), 255-259, 1995). The experiments of using Bacillus spores led to a conclusion that 2-6 hour ozone fumigation was practically effective in sterilizing materials such as cages, bedding, and working clothes. In 1992, Sato et al. reported the new system combining ionizer and catalyst for the elimination of bad smells (Sato et al., Exp. Anim. 41:39-45, 1992).

The possibility of generating a cloud of droplets by means of ultrasonic waves was first reported by Wood and Lomis (Wood et al., The physical and biological effects of high frequency sound-waves of great intensity. Phil Mag 4:417-437, 1927). Ultrasonic atomization is a very effective way of generating small droplets. Two approaches are common in this context: passing the flow across a standing ultrasonic wave (Bendig, New development of ultrasonic atomizers. In: Proceedings of the 4^{th} International Conference on Liquid Atomization and Sprays Systems. The Fuel Society of Japan, 133-138, 1988) or depositing the liquid over an ultrasonic transducer. The second case originates a fine mist of droplets that are ejected from the transducer at a very low velocity.

A disadvantage of ozone treatment, however, is that ozone only kills micro-organisms at the surface. When micro-organisms exist in layers or clusters, it has a limited effect on the innermost micro-organisms. Additionally, many people find the smell of ozone unpleasant. Further, there are known health risks associated with exposure of humans or animals to ozone above certain levels for prolonged periods.

Therefore, there exists a need for a method and/or apparatus to deodorize and/or disinfect remote areas and surfaces with odorous build-up, to neutralize odors not neutralized by ozone treatments, to neutralize odors created as a by-product of the ozone treatment of an area, and to remove ozone from an area after treatment.

### SUMMARY OF THE INVENTION

The present invention provides a process to deodorize and disinfect environmental spaces. The process includes an ozone treatment followed thereafter by a nebulized odor neutralizing treatment, which also removes ozone from the areas. The ozone treatment optionally includes passing ambient air over a discharge area to generate ozone which is then carried out of a tube into the spaces to be cleaned. The discharge area may include a ceramic plate with an anode and a cathode on each side of the ceramic plate. The nebulized-disinfectant/cleaner treatment may involve nebulizing a liquid of disinfectants, surfactants, deodorants, odor neutralizers, perfumes or ozone quenching agents, or a combination thereof, to aerosols and delivering the aerosols to spaces to be cleaned. The present invention therefore provides a process to cleanse and deodorize an environmental space including deodorizing a space with an ozone treatment for a pre-selected time; and thereafter, automatically, and without human intervention, exposing the space to a nebulized liquid, having ozone quenching properties and comprising an odor neutralizing or air freshening agent, to reduce ozone levels to a level safe for human exposure and to remove residual odors.

The present invention further provides an apparatus having an air process unit, an ozone generating unit, a liquid-nebulizing unit, and an electric system unit. A retaining housing is provided. Air flow may occur through the air process unit which includes a fan, an optional air filter, and an optional air temperature sensor. Ozone is produced by the ozone generating unit and may be discharged through an outlet tube. The liquid-nebulizing unit may include a container, a nebulizing part, and a combination of sensors. The container is for the storage of a liquid. A stream of the liquid may be nebulized into aerosols by the ceramic piezo element located within the nebulizing part. Preferably, the aerosols are in the size from 1 to 5 um. Through the forces of the air flow generated by the air process unit, both the ozone and the aerosol may be carried out by the apparatus to spaces desired to be cleaned.

The ozone treatment may additionally include: passing ambient air over a discharging area to generate ozone; and transporting said ozone into the space to be cleaned.

The discharging area may include a ceramic plate with an anode and a cathode on each side of said ceramic plate to facilitate voltage generation.

The nebulized liquid treatment may include: nebulizing a liquid to an aerosol; and delivering said aerosol to the space to be cleaned.

A component of said liquid is one of a disinfectant, a fragrance, an ozone quenching agent, an odor-neutralizing agent and any combination thereof.

The process may further include exposing the space to a nebulized disinfectant/cleaning liquid either before or after the step of deodorizing the space with an ozone treatment.

The process may further include the step of detecting the presence of a liquid provided to a liquid-nebulizing unit prior to beginning ozone production.

The process may further include the step of preventing activation of an ozone-generating unit when the presence of the liquid is not detected.

The process may further include the step of detecting an amount of liquid present in the liquid nebulizing unit.

The process may further include the step of preventing the production of ozone when the amount of liquid detected is less than a predetermined minimum amount.

The process may further include the step of preventing the production of ozone when the amount of liquid detected is more than a predetermined maximum amount.

The apparatus of the present invention for cleansing an environmental space, includes:

a housing;

an air flow source to create an air flow;

an ozone-generating unit located within said housing, to produce ozone, which is transported to said environmental space through said air flow;

a liquid-nebulizing unit located within said housing to produce aerosols which are transported to said environmental space through said air flow.

The air flow source may include a fan. The air flow source may include an air temperature sensor. The air flow source may be integral with said housing. The nebulizing area may include a ceramic piezo element.

The ozone-generating unit may include:

a ceramic plate having an anode and a cathode to facilitate voltage generation; and

a transit tube in which said ozone is carried to said environmental space.

The liquid-nebulizing unit may include:

a container for storing a liquid;

a nebulizing area located within said container for generating an aerosol from a stream of said liquid; and

a sensor located on said container to monitor the efficiency of generating said aerosol.

The apparatus may further include a liquid sensor for detecting the presence of, or the amount of, liquid in the container.

The apparatus may include a path, along which air flow generated by the air flow source flows, passing through the ozone generating unit and the liquid nebulizing unit, in any order, to the environment to be treated. The path may pass through the air source, the ozone generation unit and the liquid nebulizing unit in any order, but it can be beneficial to have the air flow source at the beginning of the path. The path may subsequently pass through the ozone generating unit and the liquid nebulizing unit in any order. Placing the ozone generating unit before the liquid nebulizing unit in the flow path can be beneficial.

The apparatus according to claim 20, may further include a transit tube arranged to prevent nebulized liquid over a certain droplet size from exiting the unit to the environmental space to be treated.

The apparatus may include a valve for preventing exposure of the liquid to the ozone generated during the ozone generation cycle. The valve may be provided in the nebulizing unit.

The ozone-generating unit may be configured to generate a maximum concentration of 0.8 ppm in the environment to be treated after the ozone generation step.

The environment to be treated may have a volume substantially comparable to the interior of an automobile.

The apparatus may be configured to carry out the method of deodorizing a space with an ozone treatment for a pre-selected time; and thereafter, automatically and without human intervention, exposing the space to a nebulized liquid, having ozone quenching properties and comprising an odor neutralizing of air freshening agent, to reduce ozone levels to a level safe for human exposure and to remove residual odors and may optionally further be arranged for carrying out an additional method of nebulizing a liquid according to any one of composition 2 or composition 3 described later in this description.

The volume of the environment to be treated may be between around 1.5 m³ and 4.5 m³.

The container may contain a liquid mixture including a combination of an odor neutralizing agent and an ozone quenching agent.

A component of said liquid mixture may be selected from one of a disinfectant, a surfactant, a complexing agent, a fragrance, an ozone-quenching agent, an odor neutralizing agent and a combination thereof.

A first composition is provided for use in ozone quenching including:

one or more surfactants;

one or more complexing agent(s); and

a component capable of neutralizing the smell of odor molecules.

The first composition may further include any one or a combination of: a suitable fragrance and one or more inorganic and/or organic solvents to obtain the specific properties for nebulizing the formulation with the apparatus. The first composition may be provided for use in the method of the present invention.

A second composition is further provided for us in disinfecting, cleaning and air freshening an environmental space, including:

one or more surfactants;

one or more complexing agent(s); and

a component with biocidal activity.

The second composition may further include one or all of: a suitable fragrance; one or more inorganic and/or organic solvents, added to obtain the specific properties suitable for nebulizing the liquid within the apparatus.

A third composition is provided for use in disinfecting, cleaning and air freshening an environmental space, including:

one or more surfactants or soaps or a combination thereof;

one or more complexing agent(s).

The third composition may further include one or all of a suitable fragrance and one or more inorganic and/or organic solvents to obtain the specific properties for nebulizing the preparation with the apparatus.

### BRIEF DESCRIPTION OF THE DRAWINGS

FIG. 1 is a perspective cut-away view of the interior of a cleansing apparatus.

FIG. 2 is a schematic view of the interior components of an apparatus.

FIG. 3 is a perspective view of an electrical control panel of the apparatus.

### DETAILED DESCRIPTION OF THE INVENTION

The present invention has utility as a process and an apparatus to cleanse and deodorize environmental spaces. In particular, it relates to the deodorization, disinfection and neutralization of odor-generating substances such as those produced by bacteria, fungi, cooking fumes, animal smells or other odors and the like, which may additionally form multi-layers or exist in hard-to-reach areas.

Traditional deodorizing treatments using ozone alone may in some circumstances be insufficient due to insufficient penetration properties of ozone in the environment to be treated. Therefore, the interior of odorous substances or clusters may be protected from the deodorizing effect of the ozone. In addition, while ozone attacks micro-organisms at their surface, the debris of cells is left behind and can form a breeding ground for new infestation. Due to the limited time during which ozone may be present in an area, either for safety reasons, or due to its natural quenching once its production is stopped, further contamination, growth of microbiological contamination may occur soon after treatment. Further, ozone itself can leave an unpleasant odor in the environment which has been treated.

The present invention improved on conventional techniques to eliminate odors, to optionally disinfect contaminants in environmental spaces, and to further neutralize odors after ozone treatment. This is achieved through a two-step process where an ozone treatment is followed by a nebulized-odor neutralizing treatment, with the latter involving the use of various odor neutralizing chemicals. The two-step process is optionally repeated until a desired level of cleansing and odor removal from the treated environment is obtained. The ozone treatment step aims to oxidize odorous substances; while the odor neutralization step has been found to provide a surprising double effect of significantly decreasing the half-life of the ozone in the environment being treated. Accordingly, a two-step operation has three effects: i) oxidizing odorous substances, ii) neutralizing remaining odors and the odor of the remaining ozone, while iii) quenching the ozone levels in the environment to a level safe for human exposure.

An optional nebulized-disinfectant/cleaner treatment, either before or after the ozone treatment, treats residual cell debris or any other contaminants either present or left behind by the ozone treatment. As a result of these steps, the treated surfaces are more resistant to new contamination where new microbiological growth may otherwise appear. Therefore, the present invention overcomes the limitations associated with the use of ozone, odor neutralizers, or disinfectants alone. An apparatus arranged to carry out the methods described herein may also be arranged to carry out a treatment of nebulizing a disinfectant and/or cleaning fluid without being combined with the ozone producing cycle. An odor neutralizing substances may also be nebulized into the environment to be treated without the use of the ozone producing step of the methods described herein. Providing a single apparatus capable of carrying out any combination of the methods described herein adds the advantages of reduced hardware costs where any combination of all, or a sub-set, of the ozone-producing, odor neutralizing, ozone quenching and disinfecting cycles may be used. Accordingly, a user only need purchase a single apparatus to be enabled to carry out any or all of the above methods.

The environmental spaces which may benefit from the present invention include, but are not limited to, car interiors, caravan interiors, boats, aircrafts, trains, coaches, trucks, buses, private homes, offices, air-conditioning units, air systems, toilet areas, sports venues, and nursery rooms. The present invention is effective to remove odors including but not being limited to, smoke odor, animal odor, human odor, odors from rotting biological materials, odors emitting by bacteria and yeast.

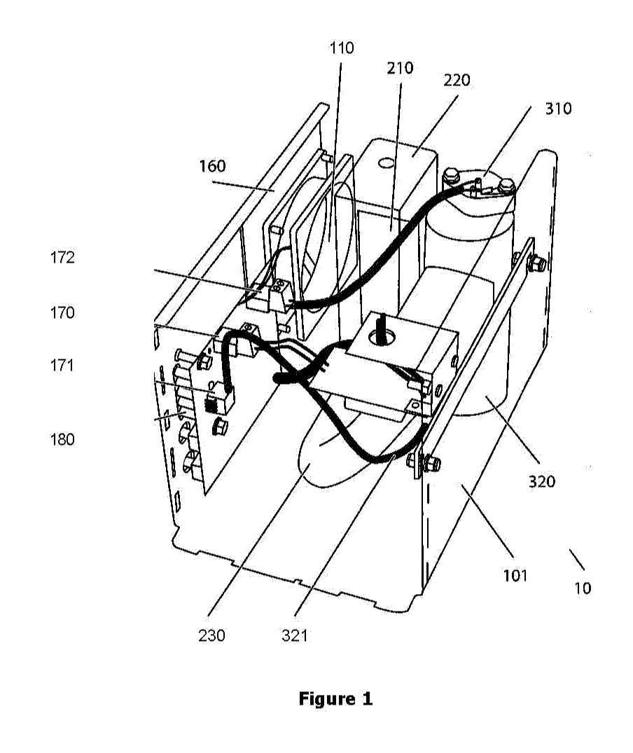

FIG. 1 illustrates an apparatus according to the present invention, arranged to carry out the method of the present invention. The apparatus is viewed from below upside-down as compared to its usual orientation during use. The apparatus 10 generates a flow of ozone into an odorous atmosphere of an environmental space to be cleaned. The apparatus includes a housing 101, a fan 110 located in the housing, an ozone generating unit including an enclosure in the form of a box 220, and a ceramic plate 210. A transit tube 230 connects the ozone generating unit to a container 320 which is equipped with high frequency ceramic transducer 310 for nebulizing a liquid within the container.

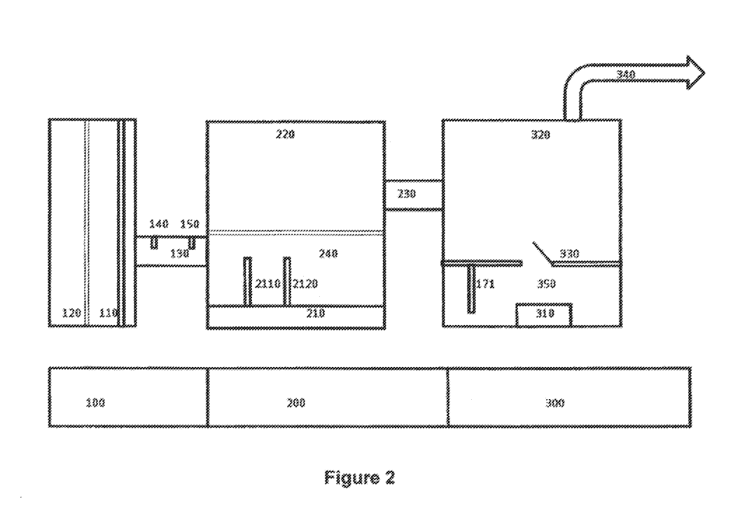

FIG. 2 illustrates schematically the arrangement of the components used to carry out the described method. Ambient air is passed between a discharge area 240 located within a box 220. The box 220 is made of metals, plastics or other durable materials. Preferably, the box 220 is made of ABS plastic or better durability. The discharge area 240 includes a ceramic plate 210 with an anode 2110 and a cathode 2120 on each side of the ceramic plate 210. The discharge area 240 is located within the lower part of the box 220. The ceramic plate is electronically powered to yield a voltage ranging from 3000 Volts to 4000 Volts. A preferred working voltage for the discharge area 240 is 3000 Volts at 60 KHz. A transit tube 230 optionally made from stainless steel is connected to the upper part of the box 220 to carry the generated ozone into the spaces desired to be cleaned through an open space with a container 320 and an outlet tube 340. An optional valve 330 is located within the container 320. The valve 330, if present, is closed during the phase of ozone generation so that the liquid contained below the valve 330 is not mixed with the ozone coming through the transit tube 230.

Liquids nebulized within a liquid-nebulizing unit 300 follow to further clean and/or deodorize and/or disinfect areas pre-treated by the ozone flow. Prior to the ozone treatment, a liquid having odor neutralizing and deodorizing properties is added to the container 320. A liquid presence detector is provided to detect the presence of liquid in the container 320. The liquid is stored to a level below the valve 330. The container 320 is preferably manufactured out of stainless 316L, which is chemically polished to have a very smooth surface. A nebulizing area 350 containing a ceramic piezo element 310 is located within the lower part of the container 320. Nebulization starts when the ceramic piezo element 310 is brought to its resonance frequency in the range of 0.5 MHz to 5 MHz and preferably at 1.7 MHz, by high frequency generator 172, attached to a microcontrol data processor 170. A dose of the liquid is emptied from the container 320 through the valve 330. At the frequency of 1.7 MHz, the ceramic piezo element 310 creates high frequency waves in the dose of the liquid, pushing the liquid up in a fountain-like column. At the edge of the fountain, the liquid breaks up into very small aerosols. The aerosols are then carried out to the spaces needing to be cleaned through the outlet tube 340. The dimensions and the shape of the outlet tube 340 are designed to facilitate the creation of an optimum airflow and to eliminate aerosols that are too large in diameter from leaving the apparatus 10. One or more sensors may be placed at critical levels on the outer wall of the container 320 so as to facilitate the maintenance of an optimal level of liquid levels within the container 320 that is the most preferred for the process of nebulization. The sensor for detecting liquid levels may take the form of a pressure sensor 171 connected to the container 320, either directly or via an optional tube 321, for detecting a pressure produced for a certain level of liquid in container 320. One or more alternative sensors may be installed to indicate when the liquid level within the container 320 is too high, too low, and when the temperature of the fluid is too high. When too little liquid is in the container, insufficient liquid may be present to properly quench ozone levels to a level safe for human exposure after the ozone generation step. The minimum amount may therefore be the minimum amount sufficient to quench ozone levels in the treated environment to a level safe for human exposure. Accordingly, a minimum and a maximum amount of fluid in the container can be defined such that the apparatus will not create ozone unless the amount of liquid in the container is above the minimum amount and below the maximum amount. In a particular example, the minimum amount may be 60 ml of the fluid having ozone quenching properties, in the particular application to automobile interiors having a volume in the range of 1.5 m³ to 4.5 m³, but the application of the method is not limited to these amounts. The physical-chemical properties of the applied chemical mixture are that the liquid is nebulized in particles in the range of 1 to 5 um.

The airflow necessary to carry the ozone and the aerosols is generated through the air process unit 100, which includes a fan 110 with an optional built-in 5 um particle filter 120. The filter 120 is replaceable from the outer side 160 of the fan 110. After passing over the particle filter, the airflow then travels through a connecting tube 130 (FIG. 2). The strength of the airflow is adjustable through modifying the operating speed of the fan 110. There is an optional air temperature sensor 140 installed on the connecting tube 130. The treatment duration of the airflow to deliver the aerosols is automatically regulated depending on the temperature of the incoming air flow. The air flow travels constantly from the connecting tube 30 through an open space within the box 220, the transit tube 230, the open space within the container 320, and the outlet tube 340.

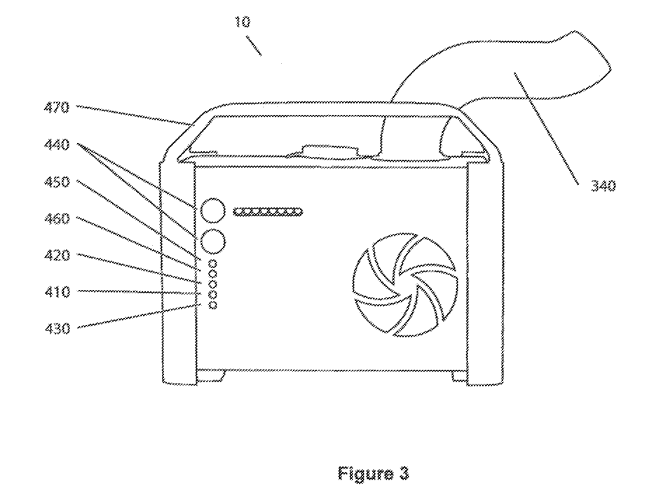

The ozone-generating unit 200, the liquid nebulizing unit 300, and the air process unit 110 are powered either by a 230 or a 110 Volt 50-60 Hz power source or a 12, 24 and 48 Volts DC power source. A general electronic system controls the speed of the fan 110, the ozone generating power of the discharging area 240 and the nebulizing power of the nebulizing area 350. In addition, the electronic system also regulates the operation of the sensors on the container 320, and the air temperature sensor 140. Located next to the air temperature sensor 140 on the connecting tube 130 is an optional heat sink 150 (FIG. 2), which may provide cooling of the electronic system. The ozone-generating unit 200 and the liquid-nebulizing unit are structurally situated so that in the case of an accidental liquid overflow, the electronics system is protected from the leaked liquid. The operation of the entire apparatus 10 is achieved with a control panel, shown in FIG. 3 and located on the outer surface of the apparatus 10, or a remote control, or the like. The control panel features a light emission diode (LED) indication of the liquid-nebulizing operation 450, liquid high level 410, liquid low level 420, liquid high temperature 430, and ready 460. In addition, duration of ozone treatment and start/stop functions are set by push buttons 440 on the control panel. With the availability of the control panel, most procedures for operating 10 are accomplished without the physical presence of a human operator so as to prevent hazardous exposure due to ozone, disinfecting chemicals, or odor neutralizing chemicals. The apparatus 10 is optionally provided with a carrying handle 470.

When the apparatus is operated to produce an ozone flow of e.g. 20 mg per hour, duration of the ozone treatment of 15 to 30 minutes is sufficient to cleanse a volume comparable to the environment of an automobile. Typical volumes for an automobile are around 2.5 m³, and may be within the range of 1.5 m³ to 4.5 m³. Once the ozone cycle, which may be a 30 minute treatment, is complete, the level is around 0.8 ppm, which is below the documented lethal dose for humans of 4 ppm. Once a liquid having odor neutralizing and ozone quenching properties is nebulized into an environment, the levels of ozone may be reduced down to less than 0.01 ppm in a very short time. This is an advantage since levels of ozone in an environment can be damaging to materials and humans as shown in the following table.

TABLE 1

| | |
|---|---|
| Health limits: | Fatal dose: 4 ppm |
| | Short term exposure: 0.1 ppm |
| | Long term exposure: 0.06 ppm |
| Material damage limits: | Rubber: 100 ppm/4 hours |
| | Fabric: 10 mg/ 30 min |
| | (=+/−12 ppm in a car) |
| Example treatment for a | Ozone generation: 0.33 mg/min = 20 mg/hour |
| volume of 2.5 m³ | Max. concentration after ozone cycle: 0.8 ppm |
| | Max. concentration after ozone + Air Purifier |
| | treatment: 0.01 ppm |

Recommended maximum exposure to ozone for humans is as illustrated in the following table, Table 2. Ozone levels safe for human exposure may therefore be less than 0.01 ppm.

TABLE 2

| | Type of Exposure | | |
|---|---|---|---|
| | Short | Long | |
| | 15 mins./day | 8 hrs/week 5 days /wk | Fatal |
| Exposure Threshold | 0.1 | 0.06 | 4 |
| (ppm) | | | |

1 ppm 03 = 2.14 mg Q/m³

Liquids particularly well suited to use in the method and apparatus of the present invention are described as follows:

Composition 1: A composition including one or more surfactants, one or more complexing agent(s), a component capable of neutralizing the smell of odor molecules, and optionally any one or a combination of: a suitable fragrance and one or more inorganic and/or organic solvents to obtain the specific properties for nebulizing the formulation with the apparatus.

The above composition has both odor neutralizing properties and ozone quenching properties and is therefore particularly well suited to use in the methods of the present invention.

A composition is particularly well suited to use in the step of exposing the space to a nebulized disinfectant/cleaning liquid, either before or after the step of deodorizing the space with an ozone treatment, or independently of that step, may be formulated as follows:

Composition 2: A composition including one or more surfactants, complexing agent(s), a component with biocidal activity, and optionally one or all of: a suitable fragrance, one or more inorganic and/or organic solvents, added to obtain the specific properties suitable for nebulizing the liquid within the apparatus.

A different liquid formulation can be particularly well suited for use is in the present invention, as an alternative to Composition 2, and in particular when emphasis is put on cleaning surfaces of environmental spaces from residual cell debris or any other contaminant present without using a biocidal active substance.

Composition 3: A composition including one or more surfactants or soaps or a combination thereof; one or more complexing agent(s) and optionally: one or all of a suitable fragrance and one or more inorganic and/or organic solvents, to obtain the specific properties for nebulizing the preparation with the apparatus.

Patent documents and publications mentioned in the specification are the levels of those skilled in the art to which the application pertains. These documents and publications are incorporated herein by reference to the same extent as if each individual document or publication was specifically and individually incorporated herein by reference.

1. A process to cleanse and deodorize an environmental space, comprising:
deodorizing a space with an ozone treatment for a pre-selected time; and
thereafter, automatically and without human intervention, exposing the space to a nebulized liquid, said liquid having ozone quenching properties and comprising an odor neutralizing or air freshening agent.

2. The process according to claim 1, wherein said ozone treatment comprises:
passing ambient air over a discharging area to generate ozone; and
transporting said ozone into said environmental space.

3. The process according to claim 1, wherein said nebulized liquid treatment comprises:
nebulizing said liquid to an aerosol; and
delivering said aerosol to said environmental space to be cleansed.

4. The process according to claim 3, wherein a component of said liquid is selected from a group consisting of: a disinfectant, a fragrance, an ozone quenching agent, an odor-neutralizing agent and any combination thereof.

5. A process according to claim 1, further comprising exposing said environmental space to a nebulized disinfectant liquid or a cleaning liquid or a combination thereof.

6. The process according to claim 1, further comprising detecting the presence of said liquid.

7. The process according to claim 6, further comprising preventing the production of ozone when the amount of said liquid detected is less than a predetermined minimum amount.

8. The process according to claim 6, further comprising preventing the production of ozone when the amount of said liquid detected is more than a predetermined maximum amount.

9. An apparatus for cleansing an environmental space, comprising:
a housing;
an air flow source to create an air flow;
an ozone-generating unit located within said housing, production of said ozone, transported to said environmental space through said air flow;
a liquid-nebulizing unit located within said housing for producing aerosols from a liquid, transported to said environmental space through said air flow.

10. The apparatus according to claim 9, wherein said air flow source comprises an air temperature sensor.

11. The apparatus according to claim 9, wherein said nebulizing area comprises a ceramic piezo element.

12. The apparatus according to claim 11, wherein said ozone-generating unit comprises:
a ceramic plate having an anode and a cathode to facilitate voltage generation; and
a transit tube in which said ozone is carried to said environmental space.

13. The apparatus according to claim 11, wherein said liquid-nebulizing unit comprises:
a container for storing said liquid;
a nebulizing area located within said container for producing said aerosol from a stream of said liquid; and
a sensor located on said container to monitor the efficiency of producing said aerosol.

14. The apparatus according to claim 13, further comprising a liquid sensor for detecting the presence of or the amount of said liquid in the said container.

15. The apparatus according to claim 9, wherein a path, along which air flow generated by the air flow source flows, passing through the ozone generating unit and the liquid nebulizing unit, in any order, to said environmental space.

16. The apparatus according to claim 13, further comprising a valve for preventing exposure of said liquid to ozone generated during the production of ozone.

17. The apparatus according to claim 9, wherein said ozone-generating unit is configured to generate a maximum concentration of 0.8 ppm after the production of ozone in said environmental space.

18. The apparatus according to claim 13, wherein a component of said liquid is selected from a group consisting of: a disinfectant, a surfactant, a complexing agent, a fragrance, an ozone-quenching agent, an odor neutralizing agent and a combination thereof.

19. The apparatus according to claim 13, wherein said liquid comprises:
one or more surfactants;
one or more complexing agent(s); and
a component capable of neutralizing the smell of odor molecules.

20. The apparatus according to claim 19, wherein said liquid comprises any one or a combination of: a suitable fragrance, and one or more solvents to obtain the specific properties for nebulizing said liquid.
